(19) United States
(12) Patent Application Publication
(10) Pub. No.: US 2016/0206778 A1
(43) Pub. Date: Jul. 21, 2016

(54) COMPOSITE SUPPORT CONTAINING SILK AND COLLAGEN, AND PREPARATION METHOD THEREOF

(71) Applicant: DONGGUK UNIVERSITY INDUSTRY-ACADEMIC COOPERATION FOUNDATION, Seoul (KR)

(72) Inventors: Jung-Keug Park, Seoul (KR); Young Kwon Seo, Seoul (KR); Mi Jung Han, Seoul (KR); Hwa Sung Lee, Seoul (KR); Su Rak Eo, Seoul (KR); Hee Hoon Yoon, Gyeonggi-do (KR); Soon Yong Kwon, Seoul (KR); Hee Jung Park, Seoul (KR)

(21) Appl. No.: 14/936,406

(22) Filed: Nov. 9, 2015

Related U.S. Application Data
(62) Division of application No. 13/634,720, filed on Nov. 30, 2012, now abandoned.

(30) Foreign Application Priority Data
Mar. 17, 2010 (KR) 10-2010-0023546

(51) Int. Cl. A61L 27/24; A61L 27/32; A61L 27/54

(52) U.S. Cl. CPC A61L 27/24; A61L 27/54; A61L 27/32; A61F 2240/001; A61L 2430/10; A61L 2430/30; A61L 2300/414

(57) ABSTRACT

Embodiments of the present invention relate to a biodegradable scaffold for replacing tissue or inducing tissue regeneration and a preparation method thereof, wherein the scaffold comprises at least one woven silk tube layer and a collagen layer inside the tube layer. The scaffold is excellent in terms of tissue regeneration and mechanical properties and causes little or no immune response after implantation. Thus, the scaffold can be effectively used as a matrix for the regeneration of ligaments and tendons and the repair of injured muscles.

### CROSS-REFERENCE TO RELATED APPLICATIONS

This application is a divisional of U.S. patent application Ser. No. 13/634,720, filed Nov. 30, 2012, entitled “Composite Support Containing Silk and Collagen, and Preparation Method Thereof” which is a 35 U.S.C. § 371 national phase application of PCT/KR2011/001841 (WO 2011/115425), filed on Mar. 16, 2011, entitled “Composite Support Containing Silk and Collagen, and Preparation Method Thereof” which application claims priority to and the benefit of Korean Patent Application No. 2010-0023546, filed Mar. 17, 2010, the disclosure of which is incorporated herein by reference in its entirety

### TECHNICAL FIELD

The present invention relates to a biodegradable scaffold for replacing tissue or inducing tissue regeneration and a preparation method thereof.

### BACKGROUND ART

In the United States, about 50 billion dollars are spent each year to treat injured ligaments of 130,000 persons or more. As various sports, including football, handball and ice hockey, have been spread, ligament injury patients have increased. In Europe, 75% of ligament injury patients required physical therapy or surgery to treat the anterior cruciate ligaments.

For the repair of injured ligaments and tendons, methods, including xenograft, allograft and autograft methods, are generally used.

The xenograft method comprises treating a bovine ligament with a chemical agent to remove the cells and grafting the treated ligament. This method was not approved by the US Food and Drug Administration (FDA), because it causes exudates, graft failure and synovitis.

Moreover, the allograft method comprises freezing the ligament of another person to remove the cells and then grafting the ligament This method suffer from various problems, including immune rejection, inhibition of ligament tissue regeneration, infection with disease, and lack of donors.

The most general therapeutic method is an autograft method of grafting the patellar tendon or semitendinous tendon of the patient himself. It is highly effective, and thus is generally used for ligament reconstruction. This therapeutic method also suffers from various problems, including the pain of donor sites, muscle atrophy, and the need for long-term rehabilitation.

To replace the biological implants, the development of non-degradable artificial synthetic ligaments has been made. A variety of artificial ligaments have been developed and implanted, but the results of observation during 15 years following implantation indicate that 40-78% of the implants cause side effects, including re-rupture, laxity, and inflammation. This is known to be because of the low abrasion resistance of the valleys or gaps between twisted yarns, axial splitting caused by bending and twisting, and structural changes caused by infiltration of other tissues.

In order to overcome such shortcomings, studies on the use of biocompatible silk for the regeneration of ligaments have been conducted.

Silk extracted from Bombyx mori (Linne) is suitable as a biomaterial, because it causes a very weak immune response in vivo, like collagen, after a process of removing sericin from silk was carried out Silk starts to be degraded after about 6 months in vivo, loses its tensile strength after 1 year, and is completely degraded within 2 years.

Accordingly, the present inventors have conducted studies to develop a biodegradable scaffold for inducing tissue regeneration, which has reduced concerns about immune responses upon implantation, is very similar to biological tissues and has excellent tissue compatibility and physical properties. As a result, the present inventors have found that a scaffold prepared by injecting collagen into a tube woven from silk has excellent physical properties and causes little or no immune response after implantation, thereby completing the present invention.

### DISCLOSURE

### Technical Problem

It is an object of the present invention to provide biodegradable scaffold, which has excellent physical properties and causes little or no immune response.

### Technical Solution

In order to accomplish the above object, embodiments of the present invention provide a composite scaffold including at least one silk tube layer and a collagen layer inside the tube layer.

Embodiments of the present invention also provide a method for preparing a composite scaffold, the method including the steps of:
forming at least one silk tube layer using a weaving machine;
removing sericin from the silk tube layer;
injecting collagen or a mixture of collagen and hyaluronic acid and/or glycosaminoglycan into the silk tube layer from which sericin was removed, followed by freeze-drying to form a collagen layer; and
cross-linking the collagen layer.

### ADVANTAGEOUS EFFECTS

A composite scaffold according to embodiments of the present invention is biodegradable, and thus does not require additional removal surgery. In addition, it is excellent in terms of tissue regeneration and mechanical properties and causes little or no immune response after implantation. Thus, it can be effectively used as a matrix for the regeneration of ligaments and tendons and the repair of injured muscles.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a photograph showing the appearance of a composite scaffold of Example 1.

FIG. 2A, 2B, 2C, 2D are a set of photographs showing an implantation process of Example 2.

FIG. 3A, 3B, 3C, 3D are a set of photographs showing H&E staining of the models used in Example 2. FIG. 3A and FIG. 3B show a model implanted with the composite scaffold of Example 1, and FIG. 3C and FIG. 3D show a model implanted with a scaffold comprising a collagen solution coated on the outside of a silk tube in Preparation Example 1.

FIG. 4 is a set of photographs showing an implantation process of Example 3.

FIG. 5A, 5B, 5C, 5D are a set of photographs showing H&E staining of the models used in Example 3. FIG. 5A shows the composite scaffold of Example 1, not coated with anything; FIG. 5B shows a composite scaffold having hydroxyapatite coated on the surface thereof; FIG. 5C shows a composite scaffold having BMP coated on the surface thereof; and FIG. 5D shows a composite scaffold coated with hydroxyapatite and BMP.

FIG. 6 is a set of photographs showing an implantation process of Example 4.

FIG. 7A, 7B, 7C, 7D, 7E, 7F shows a set of photographs showing H&E staining of the models used in Example 4. FIG. 7A, FIG. 7B, and FIG. 7C show the results obtained at 2 weeks after implantation (40× magnification), and FIG. 7D, FIG. 7E, and FIG. 7F show the results obtained at 8 weeks after implantation (100× magnification; FIG. 7A and FIG. 7D: a group implanted with a scaffold consisting only of a silk tube; FIG. 7B and FIG. 7E: a group implanted with the composite scaffold of Example 1; and FIG. 7C and FIG. 7F: a group having a shield layer formed by implanting the composite scaffold of Example 1, covering the implanted site by the amniotic membrane and then suturing the implanted site with suture material.

FIG. 8A, 8B, 8C, 8D are a set of photographs showing an implantation process of Example 5.

FIG. 9 is a photograph showing H&E staining of the implanted model used in Example 5.

### MODE FOR INVENTION

Embodiments of the present invention provide a composite scaffold comprising at least one woven silk layer and a collagen layer inside the tube layer.

The present inventors attempted to prepare a scaffold having excellent physical properties using silk and collagen, which have excellent biocompatibility. In this attempt, a scaffold prepared by simply mixing collagen with silk had problems in that collagen was lost after implantation and the physical properties thereof were reduced. The present inventors have conducted studies in order to solve these problems, and as a result, have found that a scaffold prepared by injecting collagen into tube woven from silk has excellent physical properties, cells well proliferate thereon, and the scaffold causes little or no immune response after implantation, thereby completing the present invention.

As used herein, the expression “causes little or no immune response” means that separate inflammatory reactions and exudates are not found after administration of an antibiotic during a general period after implantation and that engraftment to bone easily occurs.

In one embodiment of the present invention, the tube layer can be prepared by weaving silk threads into a tubular shape. If necessary, one or more silk tube layers can be used in an overlapping type.

Although the number of the silk tube layers can be suitably selected depending on the intended use, it is preferably 1-4 or 2-3.

In another embodiment of the present invention, the thickness of one silk tube layer is preferably 1-2 mm.

Because silk is a natural material, it is used as suture material after surgical operations without causing side effects. When silk is implanted into the human body, it promotes the secretion of collagen and the like and is easily attached to chondrocytes.

In one embodiment of the present invention, the silk tube layer may be a silk tube layer from which sericin was removed.

Sericin is a protein present in silk extracted from cocoons and constitutes cocoon fibers together with fibroin. Because sericin can cause inflammatory reactions in vivo, it is preferably removed by treatment with an alkaline salt. Examples of an alkaline salt that may be used in the present invention include, but are not limited to, sodium carbonate (Na₂CO₃), sodium hydroxide (NaOH) and the like.

In one embodiment of the present invention, the end of the silk tube layer may be coated with one or more selected from the group consisting of hydroxyapatite and bone morphogeneic protein.

Hydroxyapatite (HAp) is a basic calcium phosphate having a chemical formula of Ca₁₀(PO₄)₆(OH)₂. It is very similar to the inorganic component of human bone or tooth, is biologically nontoxic, and promotes osteoinduction at interfaces. Thus, it is a typical biomaterial which is widely used as a coating material for artificial implants.

When hydroxyapatite is coated on the end portion of the silk tube, which will come into contact with bone, it will stimulate the activity of bone cells and bind to collagen to promote the differentiation of stem cells into bone cells.

In another embodiment of the present invention, the hydroxyapatite particles may have a diameter of 1-1000 nm.

Bone morphogeneic protein (BMP) is used to promote osseointegration in bone junction sites, and examples thereof include, but are not limited to, BMP-2 and BMP-12.

In one embodiment of the present invention, the scaffold may further comprise a shield layer on the outside of the silk but layer.

The shield layer functions to inhibit the invasion of soft tissue cells other than ligaments and tendons and may be generally made of a material capable of inhibiting the invasion of other soft tissue cells. Specifically, it may be made of any one selected from the group consisting of, but not limited to, an amniotic membrane, a small intestinal submucosa membrane, a collagen membrane and a gelatin membrane and preferably has a thickness of 0.5-1 mm.

In one embodiment of the present invention, the collagen layer inside the silk tube layer is porous and functions to promote cell adhesion, migration and proliferation. It may be comprise collagen alone or one or more selected from the group consisting of a mixture of collagen and hyaluronic acid and a mixture of collagen and glycosaminoglycan.

Collagen that is used in the present invention may be insoluble collagen or soluble collagen. Specific examples of collagen that may be used in the present invention include, but are not limited to, those derived from mammals such as cattle, and those derived from marine organisms such as the skin of bony fishes.

Specific examples of the marine organisms include, but are not limited to, fishes having a pigment-free skin, for example, flatfishes such as a sole, Pleuromectes yokohamae, a turbot or a brill.

In one embodiment of the present invention, any one or more selected from the group consisting of hyaluronic acid and glycosaminoglycan may be added to the collagen which is used in the present invention.

Hyaluronic acid is an acidic mucopolysaccharide composed of a chain of alternating acetylglucosamine and glucuronic acid molecules and is widely distributed in the connective tissues of mammals together with chondroitin sulfate. Also, it is known to form in tissue a gel-like matrix, which maintains cells, makes the skin smooth and soft and protects the skin from external force and bacterial infection.

In another embodiment of the present invention, the hyaluronic acid preferably has a molecular weight of 180-350.

Glycosaminoglycan acts as a crosslinker between collagen molecules. Specific examples of glycosaminoglycan which may be used in the present invention include, but are not limited to, chondroitin, chondroitin sulfate, heparan, heparin sulfate, and dennatan sulfate.

In one embodiment of the present invention, the diameter of the composite scaffold maybe 5-10 mm or 5-7 mm.

In one embodiment of the present invention, the composite scaffold may be used for the treatment of ligament, muscle and tendon tissues.

Embodiments of the present invention also provide a method for preparing a composite scaffold, the method comprising the steps of: forming at least one silk tube layer using a weaving machine; removing sericin from the silk tube layer; injecting collagen or a mixture of collagen and hyaluronic acid/or glycosaminoglycan into the silk tube layer from which sericin was removed, followed by freeze-drying to form a collagen layer; and cross-linking the collagen layer.

Hereinafter, each step of the preparation method will be described in detail.

In the step of forming the silk tube layer, a process of weaving silk threads into a tubular shape using a weaving machine is carried out. The silk tube can be prepared to have various diameters, and a silk tube having a multilayer structure can be prepared by inserting a silk tube of a smaller diameter into a silk tube of a larger diameter.

In the step of removing sericin, a process of boiling the silk tube layer together with an alkaline salt in water for 3-10 hours, 5-9 hours or 6-8 hours is carried out

Herein, the alkaline salt is preferably used at a concentration of 0.01-0.1 M, 0.01-0.07 or 0.01-0.05 M. Specific examples of the alkaline salt that is used in the present invention include, but are not limited to, sodium carbonate (Na₂CO₃), sodium hydroxide (NaOH) and the like.

In one embodiment of the present invention, the preparation method may further comprise, after the step of removing sericin, a step of coating the silk tube layer with one or more selected from the group consisting of hydroxyapatite and bone morphogeneic protein.

Particles of hydroxyapatite which are used in the present invention may have a diameter of 1-1000 nm and may be used at a concentration of 0.1-1 g/ml, 0.1-0.5 g/ml or 0.5-0.2 g/ml.

The bone morphogeneic protein may be dissolved in a crosslinking agent at a concentration of 0.1-10 μg/ml, 0.1-5 μg/ml or 0.1-2 μg/ml, and the protein solution may be coated on the external end of the silk tube layer at a concentration of 100-200 ng/cm².

The bone morphogeneic protein that is used in the present invention may be BMP-2 or BMP-12, but is not limited thereto. The crosslinking agent that is used in the present invention may be any one or more selected from the group consisting of diphenylphosphoryl azide, glutaraldehyde, hexamethylene isocyanate, succinimide, carbodiimide, genipin, and a grape seed extract

For coating, any method may be used as long as it is a method for coating a surface with a material. Specifically, spraying, drying following precipitation and the like may be used, but is not limited thereto.

In the case in which the silk tube layer is coated with hydroxyapatite together with bone morphogeneic protein, the coating process is preferably carried out in the following manner in order to minimize the loss of the bone morphogeneic protein. Nanohydroxyapatite is first coated on the silk tube layer and dried at 2 to 50° C., 2 to 40° C. or 2 to 25° C. for 24-60 hours, 30-54 hours or 36-48 hours, and then the bone morphogeneic protein is coated thereon and dried under the same conditions as above.

In the step of forming the collagen layer, collagen is injected into the silk tube layer. Collagen which is used in the present invention may be collagen alone or a mixture of collagen and hyaluronic acid and/or glycosaminoglycan.

More specifically, the step of forming the collagen layer comprises the steps of: placing the silk tube layer in a mold (silicon tube); injecting collagen alone or the mixture of collagen and hyaluronic acid and/or glycosaminoglycan into the silk tube layer; and freeze-drying the resulting structure at a temperature of −50 to −80° C.

More specifically, in the case in which the mixture of collagen and hyaluronic acid and/or glycosaminoglycan is used, the step of forming the collagen layer may be performed by dissolving collagen in an acidic solution at a concentration of 0.1-30 mg/ml or 0.5-20 mg/ml to prepare a gel-like solution, and adding any one or more of hyaluronic acid and glycosaminoglycan thereto and injecting the mixture into the silk tube layer. Alternatively, the step may be performed by injecting collagen dissolved in an acidic solution into the silk tube layer, and then injecting any one or more of hyaluronic acid and glycosaminoglycan thereto and injecting the mixture into the silk tube layer. Alternatively, the above two method may be used in combination.

Herein, the hyaluronic acid may be used at a concentration of 0.1-30 mg/ml, 1-20 mg/ml or 7-20 mg/ml, and the glycosaminoglycan may be used at a concentration of 0.1-20 mg/ml, 1-8 mg/ml or 3-7 mg/ml.

As the acidic solution, a 0.001-0.01 M aqueous solution of acetic acid or hydrochloric acid may be used.

The injection and freeze-drying step may be performed repeatedly, if necessary, and may be repeated 1-6 times or 3-5 times, but is not limited thereto.

The crosslinking can be physically or chemically performed. The physical crosslinking can be performed by heat drying at a temperature of 100-150° C. or 100-130° C. for 24-96 hours, 36-90 hours or 60-84 hours or by UV irradiation at 4-25° C. at a power of 5-20 W for 2-10.

The chemical crosslinking can be performed by treatment with a crosslinking agent at 4-25° C. for 12-24 hours. The cross-linking agent that is used in the present invention may be any one or more selected from the group consisting of diphenylphosphoryl azide, glutaraldehyde, hexamethylene isocyanate, succinimide, carbodiimide, genipin, and a grape seed extract, but is not limited thereto.

In one embodiment of the present invention, the preparation method may further comprise, after the crosslinking step, a step of forming a shield layer.

The shield layer functions to inhibit the invasion of soft tissue cells other than ligaments and tendons and may be generally made of a material capable of inhibiting the invasion of other soft tissue cells. Specifically, it may be made of any one selected from the group consisting of an amniotic membrane, a small intestinal submucosa membrane, a collagen membrane and a gelatin membrane, but is not limited thereto.

In another embodiment of the present invention, the shield layer can be formed by covering the composite scaffold with the already prepared membrane-type matrix and then applying fibrin glue thereto.

In still another embodiment of the present invention, the shield layer can also be formed by dipping the crosslinked composite scaffold in any one polymer solution selected from the group consisting of collagen solution, hyaluronic acid solution, chitosan solution, alginate solution, polylactic acid (PLA) solution, polyglycolic acid (PGA) solution and polycaprolactam (PCL) solution, and then drying the composite scaffold in air.

In still another embodiment of the present invention, the shield layer can also be formed by electrospinning any one polymer solution selected from the group consisting of collagen solution, hyaluronic acid solution, chitosan solution, alginate solution, polylactic acid (PLA) solution, polyglycolic acid (PGA) solution and polycaprolactam (PCL) solution onto the crosslinked composite scaffold to form a film.

### EXAMPLES

Hereinafter, the present invention will be described in detail with reference to examples. It is to be understood, however, that these examples are for illustrative purposes only and are not intended to limit the scope of the present invention. The examples of the present invention are provided to further completely explain the invention to those of ordinary skill in the art.

### Preparation Example 1

### Preparation of Silk Tube Layer

Silk (Won Corporation, Korea) was woven into tubes having diameters of 2 mm and 4 mm, respectively, using a weaving machine. Then, the smaller-diameter silk tube was inserted onto the larger-diameter silk tube to form a double-layer tube. Then, to remove sericin, the tube was treated with a 0.02M Na₂CO₃ solution at 100° C. for 8 hours. Then, the tube was clean with 0.3% ivory cleaner, washed with triple-distilled water for 3 days, and dried at 4° C., thereby preparing a silk tube layer from which sericin has been removed.

### Example 1

### Preparation of Composite Scaffold

Atelocollagen (Bioland, Korea) was dissolved in a 0.05 M aqueous solution of hydrochloric acid to prepare 20 mg/ml of a gel-like solution. To the gel-like solution, 20 mg/ml of chondroitin sulfate (Sigma, USA) was added in an amount of 10 parts by weight based on 100 parts by weight of the gel-like solution to prepare a mixed solution of collagen and chondroitin sulfate.

The mixed solution was injected into the double-layer silk tube prepared in Preparation Example 1. Specifically, the double-layer silk tube was placed in a silicon tube (mold), and the mixed solution was injected therein, followed by freeze-drying at −80° C.

The injection and freeze-drying step was repeated four times, and then 20 mg/ml hyaluronic acid (NovaMatrix, Norway) was injected into the tube, followed by freeze-drying at −80° C. Then, the tube was subjected to cross-linking using a carbodrimide crosslinking agent for 20 minutes, after it was washed and freeze-dried, thereby preparing a composite silk scaffold.

Then, the composite scaffold was sterilized by 10 kGy of γ-irradiation and stored at −20° C. until use.

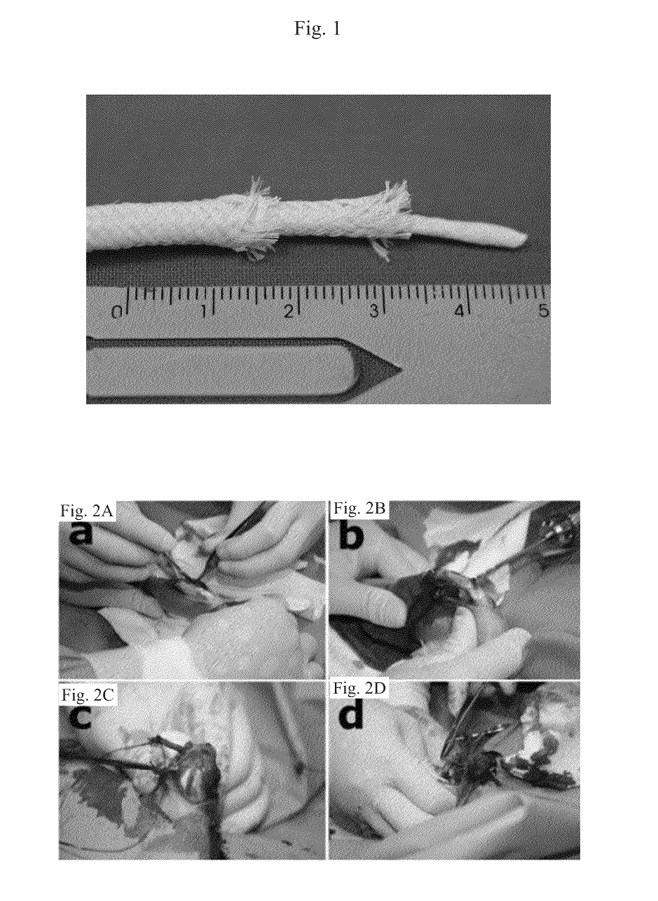

FIG. 1 shows the structure of the prepared composite scaffold. As can be seen in FIG. 1, the composite scaffold comprises two silk tube layers and a collagen layer inside the silk tube layers.

### Example 2

### Evaluation of Effect of Composite Scaffold in Animals

To evaluate the effect of the composite scaffold for ligaments in animals, rabbits with collateral ligament injury were used.

As shown in FIG. 2, a patellar tendon site was removed from rabbits (FIG. 2a), after which the collateral ligament of the knee was removed and then a tunnel was formed in the condyle portion using a 2.5-mm drill (FIG. 2b). A scaffold, prepared by coating a collagen on the outside of the silk tube of Preparation Example 1, or the composite scaffold of the present invention, was inserted into the formed tunnel in a ‘ [symbol] ’ shape (FIG. 2c), and then the tunnel portion was sutured in a ‘ [symbol] ’ shape (FIG. 2d). After the surgical operation, an antibiotic was administered to the rabbits for about 10 days.

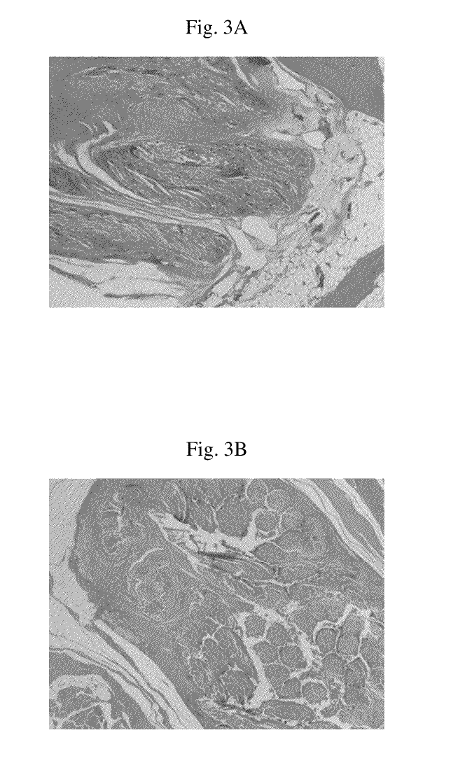

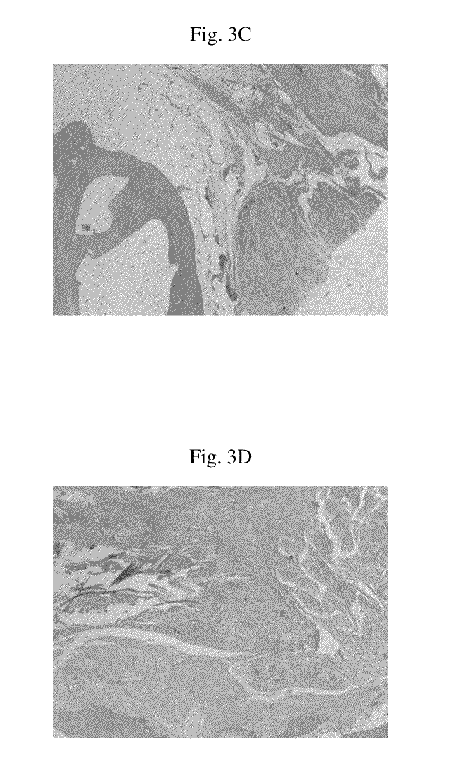

6 months after the implantation, the animal model with collateral ligament injury was subjected to histological examination in order to examine the biocompatibilities of the scaffold, comprising the collagen solution coated on the outside of the silk tube of Preparation Example 1, and the composite scaffold of the present invention. The results of the histological examination are shown in FIG. 3.

As can be seen in FIG. 3, in both the scaffold, comprising the collagen solution coated on the outside of the silk tube of Preparation Example 1 (FIGS. 3c and 3d), and the composite scaffold of the present invention (FIGS. 3a and 3b), no sign of inflammation was observed and no implant failed. However, it could be seen that, in the group implanted with the composite scaffold of the present invention (FIGS. 3a and 3b), engraftment to bone more easily occurred.

### Example 3

### Evaluation of Effect of Composite Scaffold in Animals

To evaluate the effect of the composite scaffold for ligaments in animals, rabbits with collateral ligament injury were used.

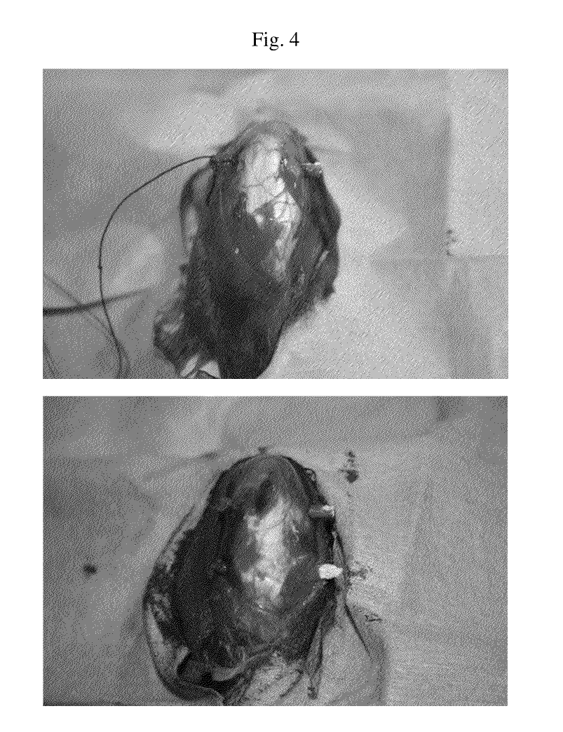

As shown in FIG. 4, the skin of rabbits was incised, and then a tunnel was formed in the condyle portion using a 2.5-mm drill. a) The composite scaffold of Example 1, not coated with anything, b) the composite scaffold having hydroxyapatite coated on the surface thereof, c) the composite scaffold having BMP coated on the surface thereof, or d) the composite scaffold coated with hydroxyapatite and BMP was inserted into the formed tunnel. After the surgical operation, an antibiotic was administered to the rabbits for about 10 days.

6 Months after the implantation, the degree of osseointegration was observed.

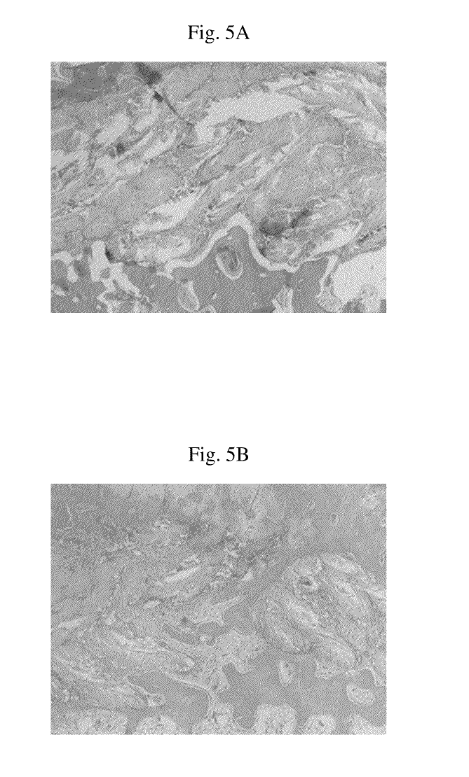

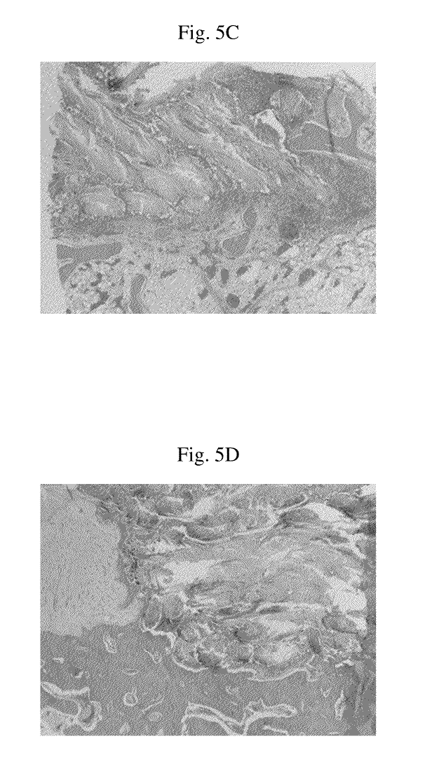

The results of the observation are shown in FIG. 5. As can be seen therein, larger amounts of calcium deposits were observed in the composite scaffold having BMP coated on the surface (c) and the composite scaffold (d) coated with hydroxyapatite and BMP.

### Example 4

### Evaluation of Effect of Composite Scaffold on Tendon Regeneration

To evaluate the effect of the composite scaffold for ligaments on tendon regeneration, an Achilles-tendon injury model was used.

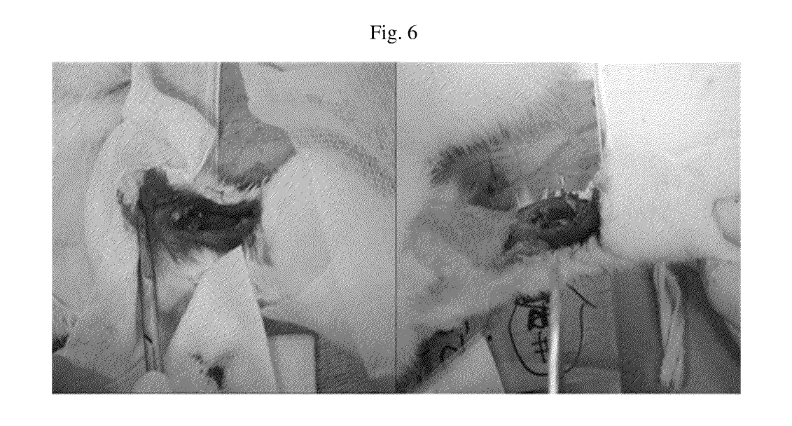

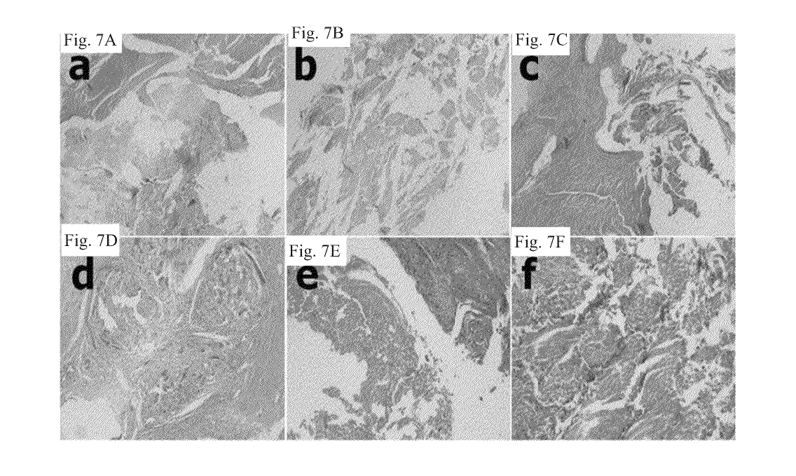

In order to evaluate the biocompatibility of the composite scaffold for ligaments, as shown in FIG. 6, the Achilles-tendon of the rabbit's hind leg was removed by about 15 mm to form a tendon injury model. Then, a scaffold consisting only of the silk tube of Preparation Example 1 or the composite scaffold of Example 1 was inserted into the tendon-removed portion, which was then sutured with suture material. Then, an antibiotic was administered to the rabbits for about 10 days.

In addition, in some of the rabbits implanted with the composite scaffold of Example 1, the implanted portion was covered with the amniotic membrane and sutured with suture material, thereby forming a shield layer.

Then, the rabbits were subjected to histological examination, and the results of the examination are shown in FIG. 7.

FIGS. 7a, 7b and 7c show the results obtained 2 weeks after the implantation (4th magnification), and FIGS. 7d, 7e and 7f show the results obtained 8 weeks after the implantation (10th magnification).

As can be seen in FIG. 7, in the group implanted with the scaffold consisting only of the silk tube of Preparation Example 1 (FIGS. 7a and 7d), slight signs of inflammation were observed, but in the group implanted with the composite scaffold of Example 1 (FIGS. 7b and 7e), no inflammatory reaction was observed, and engraftment easily occurred. In addition, it was shown that, in the group having a shield layer formed by implanting the composite scaffold of Example 1, covering the implanted site by the amniotic membrane and then suturing the implanted site with suture material (FIGS. 7c and 7f), the engraftment and regeneration of tissue easily occurred.

### Example 5

### Evaluation of Effect of Composite Scaffold on Tendon Regeneration

To evaluate the effect of the composite scaffold on the reconstruction of injured anterior cruciate ligaments in animals, a dog model was used.

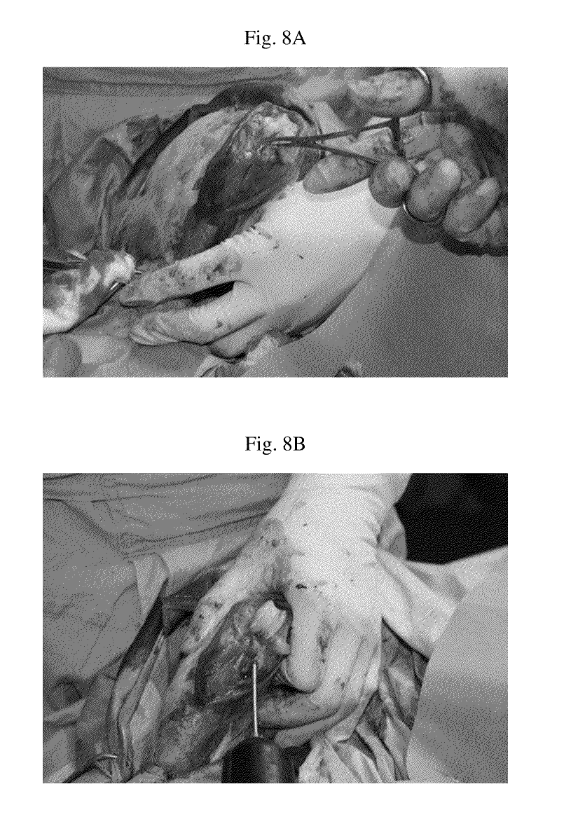

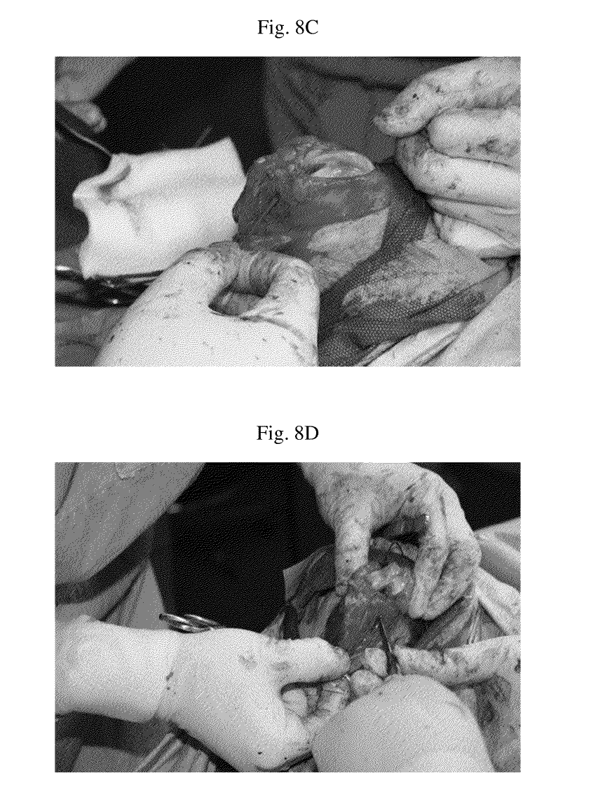

As shown in FIG. 8, muscle was removed from the patellar tendon site, and the anterior cruciate ligament of the knee was completely removed (FIG. 8a), after which a tunnel was formed at the cruciate ligament position using a 2.5-mm drill (FIG. 8b). The composite scaffold of Example 1 was inserted into the formed tunnel (FIG. 8c), after which both entrances to the tunnel were mixed with a screw (FIG. 1d).

Then, cell migration and osseointegration were observed.

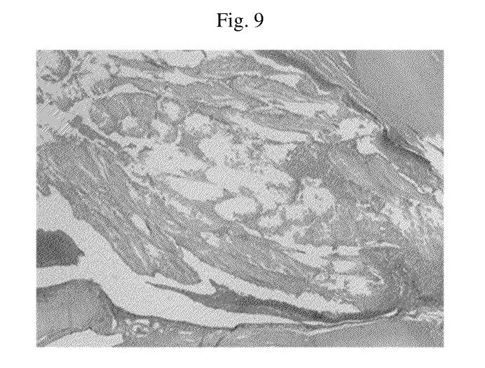

As shown in FIG. 9, slight signs of inflammation were observed, but no exudates were found, and engraftment to bone easily occurred. 6 Months after the implantation, there were no rupture and loosening, suggesting that the reconstruction of the ligament well proceeded.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A method for preparing a composite scaffold, the method comprising the steps of:
forming at least one silk tube layer using a weaving machine;
removing sericin from the silk tube layer;
injecting collagen or a mixture of collagen and hyaluronic acid/or glycosaminoglycan into the silk tube layer from which sericin was removed, followed by freeze-drying to form a collagen layer; and
cross-linking the collagen layer.

2. The method of claim 1, wherein the method further comprises, after the step of removing sericin, a step of coating the silk tube layer with any one or more selected from the group consisting of hydroxyapatite and bone morphogeneic protein.

3. The method of claim 1, wherein the method further comprises, after the step of cross-linking the collagen layer, a step of forming a shield layer.

4. The method of claim 1, wherein the woven silk tube layer has a thickness of 1-2 mm.

5. The method of claim 3, wherein the shield layer is any one selected from the group consisting of an amniotic membrane, a small intestinal submucosa membrane, a collagen membrane and a gelatin membrane.

6. The method of claim 3, wherein the shield layer has a thickness of 0.5-1 mm.

7. The method of claim 1, wherein the composite scaffold has a diameter of 5-10 mm.

8. The method of claim 1, wherein the composite scaffold is for treatment of ligament, muscle and tendon tissues.
